(19) United States
(12) Patent Application Publication
(10) Pub. No.: US 2009/0075872 A1
(43) Pub. Date: Mar. 19, 2009

(54) METHODS AND MEANS

(76) Inventor: David J. Grainger, Cambridge (GB)

Correspondence Address: SCHWEGMAN, LUNDBERG & WOESSNER, P.A., P.O. BOX 2938, MINNEAPOLIS, MN, 55402, US

(21) Appl. No.: 11/577,974

(22) PCT Filed: Oct. 25, 2005
(86) PCT No.: PCT/GB05/04122

§ 371 Date: Oct. 31, 2008

(30) Foreign Application Priority Data
Oct. 25, 2004 (GB) 0423658.4

(51) Int. Cl. C12Q 1/02; A61K 38/17; A61P 25/28; A61P 29/00; A61P 19/10; A61P 9/00; A61P 9/10

(52) U.S. Cl. 514/12; 435/29

(57) ABSTRACT

This invention relates to methods and means for the stimulation of phagocytosis and in particular to the phagocytosis of apoptotic cells, and discloses a role for the protein product of the apoE gene (apolipoproteinE) as a regular of apoptotic cell clearance. ApoE mimetics and other compounds which stimulate the clearance of apoptotic cells may be useful in the treatment of a range of disorders. One aspect of the invention provides a method of identifying and/or obtaining a compound for the treatment of a condition associated with decreased endogenous apoE activity in an individual comprising: determining the ingestion of apoptic cells by a macrophage in the presence of a test compound. An increase in apoptic cell ingestion in the presence relative to the absence of test compound may be indicative that the compound may be useful in the treatment of a condition associated with decreased endogenous apoE activity.

This invention relates to methods and means for the stimulation of phagocytosis and in particular to the phagocytosis of apoptotic cells.

ApolipoproteinE (apoE), together with apolipoproteinB, constitutes the majority of the protein present in the triglyceride-rich lipoprotein particles LDL and VLDL.

The apoE component is a ligand for the LDL receptor (LDLR) and a family of LDL-receptor-related proteins or LRPs [Herz, J. and U. Beffert. 2000. Nat Rev Neurosci 1:51]. Homozygous deletion of the apoE gene in mice has a dramatic effect on cholesterol transport, resulting in a large increase in the plasma levels of LDL and VLDL due to the failure of LDLR and LRP mediated clearance of these lipoproteins from the blood [Moghadasian, M. H. et al 2001. Faseb J 15:2623; Zhang, S. H. et al 1992. Science 258:468]. The most obvious phenotype of apoE-deficient mice is the development of vascular lipid lesions resembling early human atherosclerosis, even on a diet with normal fat content.

Three common allelic variants of the apoE gene exist in man, designated apoε2, ε3 and ε4 encoding apoE2, E3 and E4 respectively.

ApoE2 and E4 differ from the most common E3 isotype by a single amino acid substitution in each case. The presence of even a single copy of the apoε4 allele results in an increased risk of coronary heart disease compared with the wild-type apoε3 homozygote [for example, see Davignon, J., et al 1988. Arteriosclerosis 8:1; Eichner, J. E. et al. 2002. Am J Epidemiol 155:487]. The apoε2 allele is associated with complex defects in lipoprotein metabolism such as type III hyperlipoproteinemia and has been associated with both an increase and a decrease in atherosclerosis, depending on the study design. The precise molecular basis for the association between apoE genotype and cardiovascular disease incidence remains uncertain.

The presence of the apoε4 allele has been associated with increased risk of Alzheimer's disease [Corder, E. H., et al 1993 Science 261:921, Poirier, J. et al 1993. Lancet 342:697, Mahley, R. W. et al 2000. Annu Rev Genomics Hum Genet 1:507.] and increased risk of osteoporosis [Cauley, J. A. et al 1999. J Bone Miner Res 14:1175; Salamone, L. M., et al. 2000. J Bone Miner Res 15:308.]. Indeed, the associations between apoE haplotype and a range of prevalent disorders are sufficiently strong that apoE remains one of the few loci which has been unequivocally associated with lifespan: the apoε2 allele is over-represented among octogenarians suggesting that the apoE genotype contributes to longevity [Schachter, F. et al 1994. Nat Genet 6:29].

ApoE may also regulate local inflammation through receptor-mediated signaling cascades independent of lipoprotein transport. For example, apoE, as well as a 14 amino acid receptor-binding peptide that does not bind cholesterol, was able to suppress the local inflammatory response to experimental cerebral ischemia in vivo [Lynch, J. R. et al. 2001. J Neuroimmunol 114:107 and Sheng, H. et al 1998. J Cereb Blood Flow Metab 18:361]. Cell culture studies suggest this effect may be mediated through suppression of macrophage function [Laskowitz, D. T. et al 1997 J Neuroimmunol 76:70 and Laskowitz, D. T. et al 2001. Exp Neurol 167:74]. Studies of chronic infections in apoE-deficient mice lead to a similar conclusion that apoE somehow suppresses macrophage function [Van Oosten, M. et al. 2001. J Biol Chem 276:8820 and Roselaar, S. E. and A. Daugherty. 1998 J Lipid Res 39:1740. and de Bont, N. et al. 1999. J Lipid Res 40:680.].

The present inventors have discovered a hitherto unsuspected role for the protein product of the apoE gene (apolipoproteinE) as a regulator of apoptotic cell clearance. ApoE mimetics and other compounds which stimulate the clearance of apoptotic cells may be useful in the treatment of a range of disorders.

One aspect of the invention provides a method of identifying and/or obtaining a compound for the treatment of a condition associated with decreased endogenous apoE activity in an individual comprising:
determining the ingestion of apoptotic cells by a macrophage in the presence of a test compound.

Ingestion of apoptotic cells by the macrophage in the presence of the test compound may be compared with ingestion in comparable reaction medium and conditions in the absence of a test compound. An increase in apoptotic cell ingestion in the presence relative to the absence of test compound may be indicative that the compound may be useful in the treatment of a condition associated with decreased endogenous apoE activity.

In some embodiments, the macrophage may be exposed to the test compound prior to determining apoptotic cell ingestion.

A condition associated with decreased endogenous apoE activity may be genetic in origin (for example, the presence of one or more apoE4 alleles), environmental in origin (for example, reduced apoE production by cells exposed to high levels of cholesterol) or may be a disease condition selected from the group consisting of Alzheimer's Disease, atherosclerosis, stroke and osteoporosis.

In some embodiments, ingestion may be determined in the presence of ApoE. For example, a method may be performed under conditions where ApoE levels are normal, for example by using wild-type macrophages and apoptotic cells (in which ApoE is present) in buffers containing physiological levels of ApoE. In such embodiments, a test compound which is an agonist of ApoE and which increases the rate of phagocytosis relative to normal conditions may be identified. In some embodiments, a method may comprise the steps of exposing a macrophage to a test compound and contacting the macrophage with an apoptotic polypeptide.

In other embodiments, a method may be performed in the absence of apoE, for example by using ApoE deficient macrophages and apoptotic cells (for example cells derived from apoE-deficient mice) under ApoE free conditions (e.g. using medium and buffers which do not contain any apoE). In such embodiments, a test compound which replicates the function of apoE in stimulating apoptotic cell phagocytosis may be identified. A method may comprise determining the ingestion of ApoE deficient apoptotic cells by an ApoE deficient macrophage under ApoE free conditions in the presence of a test compound.

Reduced apoptotic cell clearance is shown herein to lead to a marked pro-inflammatory phenotype. Methods described herein may be used to identify compounds that have anti-inflammatory properties. Suitable compounds stimulate apoptotic cell clearance, reducing the need to recruit as many phagocytes to the inflamed tissue, reducing production of inflammatory mediators by the said phagocytes and thereby resulting in a systemically anti-inflammatory action. As a result, the methods described herein may be used to identify therapeutic agents which are useful in a wide range of diseases with an inflammatory component, even if such diseases are not clearly associated with either reduced apoE function or increased demand for clearance of cell debris.

Another aspect of the invention provides a method of identifying and/or obtaining a compound for the treatment or prevention of a condition associated with increased inflammatory activity:
determining the ability of an ApoE polypeptide to stimulate the ingestion of apoptotic cells by a macrophage in the presence of a test compound.

Stimulation of the ingestion or phagocytosis of apoptotic cells by the macrophage by the ApoE polypeptide in the presence the test compound may be compared with ingestion in comparable reaction medium and conditions in the absence of a test compound. An increase in apoptotic cell ingestion in the presence relative to the absence of test compound may be indicative that the compound may be useful in the treatment of a condition associated with increased inflammatory activity.

An ApoE polypeptide may be an ApoE polypeptide from any mammalian species, for example a mouse ApoE or a human ApoE (for example human ApoE2, ApoE3 or ApoE4) or may be a fragment or variant thereof which retains one or more activities of the wild-type protein, in particular the stimulation of apoptotic cell phagocytosis.

For example, an ApoE polypeptide may comprise an amino acid sequence which shares greater than about 30% sequence identity with human ApoE3, greater than about 40%, greater than about 45%, greater than about 55%, greater than about 65%, greater than about 70%, greater than about 80%, greater than about 90% or greater than about 95%. The sequence may share greater than about 30% similarity with human ApoE3, greater than about 40% similarity, greater than about 50% similarity, greater than about 60% similarity, greater than about 70% similarity, greater than about 80% similarity or greater than about 90% similarity.

Sequence similarity and identity are commonly defined with reference to the algorithm GAP (Genetics Computer Group, Madison, Wis.). GAP uses the Needleman and Wunsch algorithm to align two complete sequences that maximizes the number of matches and minimizes the number of gaps. Generally, default parameters are used, with a gap creation penalty=12 and gap extension penalty=4. Use of GAP may be preferred but other algorithms may be used, e.g. BLAST (which uses the method of Altschul et al. (1990) J. Mol. Biol. 215: 405-410), FASTA (which uses the method of Pearson and Lipman (1988) PNAS USA 85: 2444-2448), or the Smith-Waterman algorithm (Smith and Waterman (1981) J. Mol Biol. 147: 195-197), or the TBLASTN program, of Altschul et al. (1990) supra, generally employing default parameters. In particular, the psi-Blast algorithm (Nucl. Acids Res. (1997) 25 3389-3402) may be used. Sequence identity and similarity may also be determined using Genomequest™ software (Gene-IT, Worcester Mass. USA).

Sequence comparisons are preferably made over the full-length of the relevant sequence described herein.

Similarity allows for “conservative variation”, i.e. substitution of one hydrophobic residue such as isoleucine, valine, leucine or methionine for another, or the substitution of one polar residue for another, such as arginine for lysine, glutamic for aspartic acid, or glutamine for asparagine.

In some embodiments, an ApoE polypeptide may be a mammalian ApoE polypeptide, for example a murine ApoE having the sequence of database accession number AAH83351.1 or a human ApoE having the sequence of database accession number P02649 or NP_{—}000032.1 or a allelic variant described therein.

A fragment of a full-length sequence may consist of fewer amino acids than the full-length sequence. For example a fragment may consist of at least 10, at least 20, at least 30, at least 40, at least 50, at least 60, at least 70, at least 80, at least 90 or at least 100 amino acids of the full length sequence but 290 or less, 280 or less, 270 or less, 260 or less, 250 or less, 240 or less, 230 or less, 220 or less, 210 or less or 2000 or less amino acids.

Conditions associated with increased inflammatory activity may include multiple sclerosis, rheumatoid arthritis, irritable bowel syndrome, Crohn's Disease, stroke, myocardial infarction, asthma, allergic rhinitis, eczema, psoriasis and contact hypersensitivity dermatitis.

In some embodiments, the ApoE polypeptide may be on the surface of the macrophage and/or apoptotic cells. In other embodiments, the ApoE polypeptide may be present in the reaction medium.

In some embodiments, the ability of a test compound to stimulate the ingestion of apoptotic cells by a macrophage may be determined in the absence of ApoE. A method of identifying and/or obtaining a compound for the treatment or prevention of a condition associated with increased inflammatory activity may comprise:
determining the ingestion of one or more apoptotic ApoE deficient cells by a ApoE deficient macrophage in the presence of a test compound.

ApoE deficient cells may, for example, be obtained from an ApoE deficient mouse, which may be produced in accordance with known methods, as described herein.

Ingestion of apoptotic cells by the ApoE deficient macrophage in the presence of the test compound may be compared with ingestion in comparable ApoE deficient reaction medium and conditions in the absence of a test compound.

The accumulation of cellular debris in an individual, for example, as a result of tissue trauma (such as head injury or gun shot wounds) or rampant infectious diseases (such as bacterial sepsis, pancreatitis or pericarditis), may lead to significant medical problems.

Methods as described herein may be useful in identifying and/or obtaining a compound that stimulates apoptotic cell clearance and may be useful in the treatment of a condition associated with apoptotic cell accumulation.

A method of identifying and/or obtaining a compound for the treatment of a condition associated with apoptotic cell accumulation may comprise:
determining the ability of an ApoE polypeptide to stimulate the ingestion of apoptotic cells by a macrophage in the presence of a test compound.

Stimulation of the ingestion or phagocytosis of apoptotic cells by the macrophage by the ApoE polypeptide in the presence the test compound may be compared with ingestion in comparable reaction medium and conditions in the absence of a test compound. An increase in stimulation in the presence relative to the absence of test compound may be indicative that the compound may be useful in the treatment of a condition associated with apoptotic cell accumulation.

In some embodiments, the ApoE polypeptide may be on the surface of the macrophage and/or apoptotic cells. In other embodiments, the ApoE polypeptide may be present in the reaction medium.

Conditions associated with apoptotic cell accumulation include tissue trauma, Traumatic Brain Injury, acute respiratory distress syndrome, bacterial sepsis, pancreatitis or pericarditis.

In some embodiments, the ability of a test compound to stimulate the ingestion of apoptotic cells by a macrophage may be determined in the absence of ApoE. A method of identifying and/or obtaining a compound for the treatment or prevention of a condition associated with apoptotic cell accumulation may comprise:
contacting an ApoE deficient macrophage with one or more apoptotic ApoE deficient cells in the presence of a test compound; and
determining the ingestion of the apoptotic cells by the macrophage.

Ingestion of apoptotic cells by the ApoE deficient macrophage in the presence of the test compound may be compared with ingestion in comparable ApoE deficient reaction medium and conditions in the absence of a test compound.

Macrophages are large mononuclear phagocytic cells which are present in blood, lymph, and other tissues and ingest foreign material and cell debris, including apoptotic cells. Macrophages are involved in both specific and non-specific immune responses.

A macrophage for use in the present methods may be a cultured macrophage. Cultured macrophages may be obtained by any one of a range of suitable methods, including, for example, in vitro differentiation of freshly prepared human peripheral blood monocytes (such as by exposure to phorbol esters); in vitro differentiation of monocytic cell lines, such as the human myelomonocytic cell line THP-1; and preparation of peritoneal macrophages by washing the peritoneum of an animal (such as a mouse or a rat) with sterile buffer and culturing the resultant peritoneal exudate.

An apoptotic cell is a cell that is undergoing or has undergone apoptosis or programmed cell death. Apoptotic cells for use in the present methods are preferably mammalian cells and may be from the same tissue, from the same organism or same species of organism as the macrophages or may be from a different tissue, organism and/or species. Conveniently, apoptotic thymocytes may be used in the present methods.

A range of suitable methods for obtaining apoptotic cells are known in the art, including, for example, withdrawal of serum from cultured primary lymphocytes, typically thymocytes; treatment of cultured cells with apoptosis inducers such as Fas ligand or Granzyme B; and isolation of cells undergoing apoptosis in vivo using selectable cell surface markers of apoptotic induction (such as Annexin V binding). Typically, more than 50% of the cells in the population to be used will be undergoing apoptosis, as defined, for example, by staining with labelled Annexin V, or by staining for active Caspase 3.

Ingestion of apoptotic cells by the macrophage may be determined by any convenient method. For example, a fixed number of apoptotic cells may be added to the macrophage or macrophage population, for example in replicate wells of a multi-well plate, ranging from 0.1 apoptotic cell per macrophage to 1,000 apoptotic cells per macrophage, typically from 1 to 100 apoptotic cells per macrophage. In preferred embodiments, the test compound is present in the culture medium throughout the time when phagocytosis is occurring.

Macrophages may be incubated with the apoptotic cells for a defined period of time during which phagocytosis occurs. Typically, the incubation is performed at a temperature between 25° C. and 40° C., more typically at 37° C. The duration of the incubation may be selected so that a detectable number of apoptotic cells have been taken up, but less than 50% of the total apoptotic cells added have been taken up (so that the availability of apoptotic cells has not become limiting for further phagocytosis). This may take, for example, between 10 minutes and 10 hours at 37° C., more typically between 30 minutes and 2 hours.

In some embodiments, the number or proportion of apoptotic cells which have been taken up into the macrophages by phagocytosis may be determined. This may be conveniently achieved by detecting the apoptotic cells that were added to the macrophages and distinguishing apoptotic cells outside, or on the external surface of, the macrophages from apoptotic cells inside or on the internal surface of the macrophages.

Apoptotic cells may be detected by any suitable method known in the art and is conveniently achieved by pre-labelling the cells prior to induction of apoptosis. A range of suitable labels and dyes are available commercially, including Cell Tracker Green (Molecular Probes Inc.). The labelled apoptotic cells may then be identified and counted by detecting the label under appropriate conditions, for example by microscopy.

Internal and external apoptotic cells may be distinguished by washing the macrophages under conditions in which apoptotic cells which are outside, or stuck to the external surface of, the macrophages are dislodged and discarded, while apoptotic cells which are inside, or stuck to the internal surface of, the macrophages are retained. Any suitable washing procedure which dislodges external but not internal apoptotic cells may be employed including for example a series of washes with ice cold Dulbecco's PBS. Following washing, the number of ingested apoptotic cells may be counted, together with the total number of macrophages performing the phagocytosis.

The rate of phagocytosis may be determined from the extent of phagocytosis occurring in a defined period and may be conveniently expressed as the number of apoptotic cells (e.g. thymocytes) taken up per macrophage per hour. Typically, this value ranges from 0.1 to 100 in wild-type cells in the absence of a phagocytosis stimulator. For example, for murine peritoneal macrophages ingesting apoptotic thymocytes, this value may range from 2 to 4 and for apoE-deficient macrophages ingesting apoE deficient thymocytes, this value may range from 0.5 to 1.

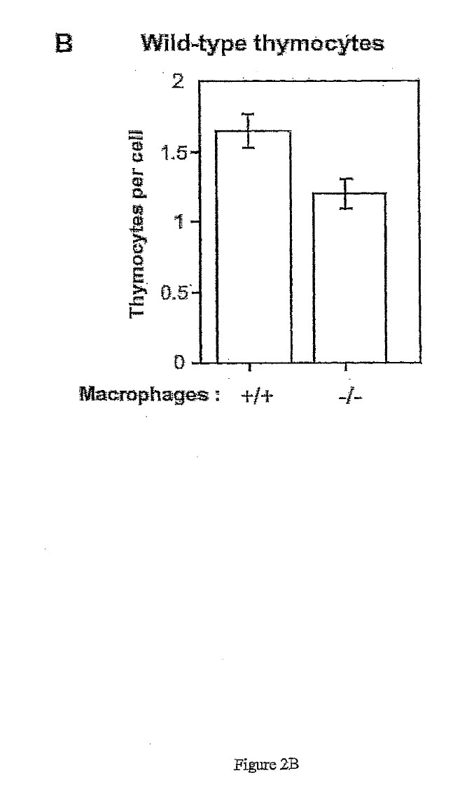

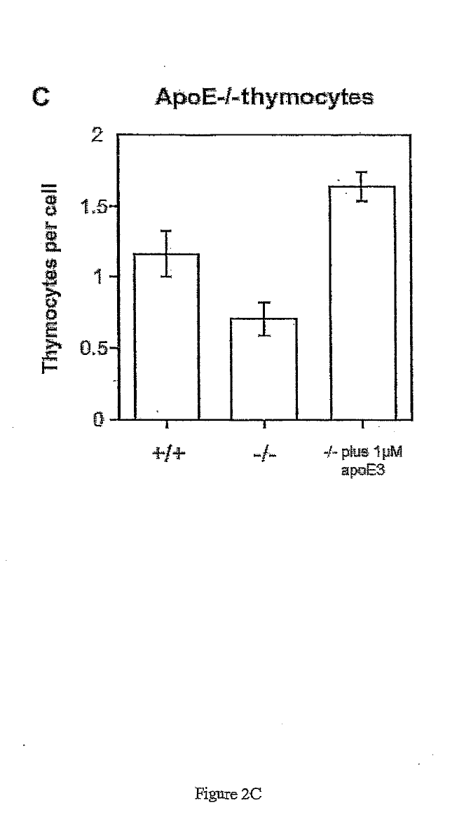

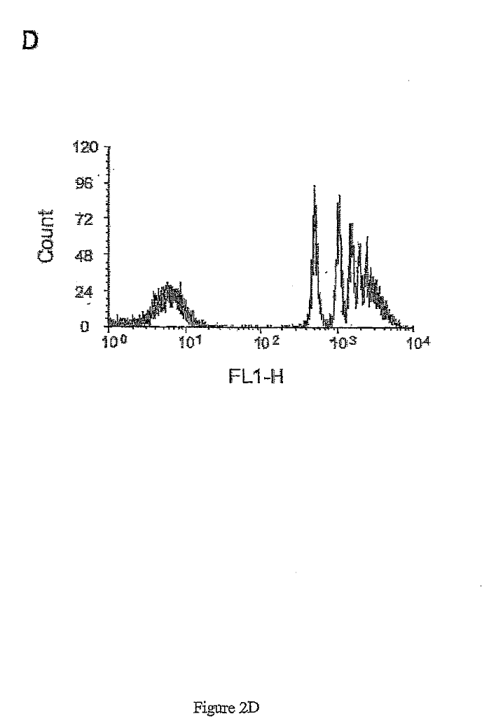

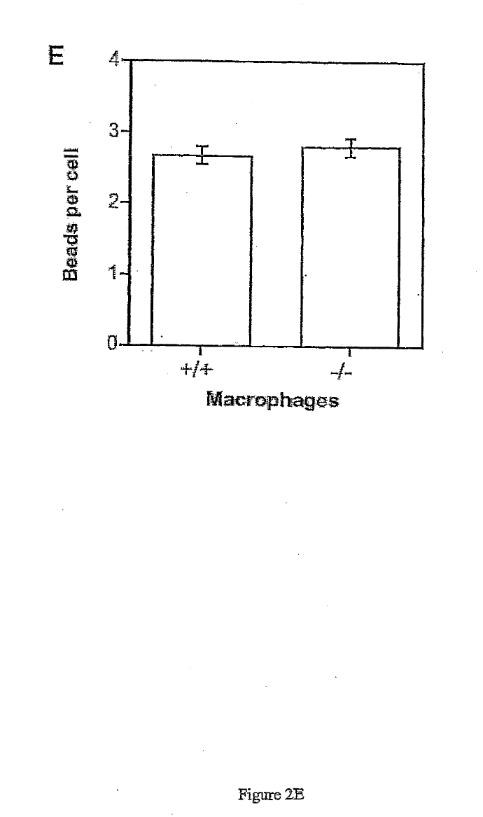

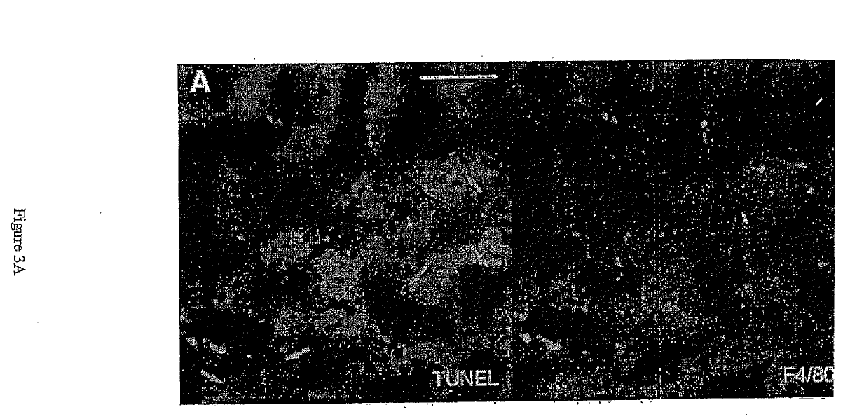

The effect of the test compound may be expressed as the fold-change in this value compared to control cells not exposed to the compound. A typical response, using recombinant human apoE as the test compound is shown in FIG. 2.

The test compound may be added at a suitable concentration, which is normally determined by trial and error depending upon the type of compound used, but is typically between 10 pM and 10 mM, more typically between 1 nM and 100 μM. The test compound may be added in any suitable biologically compatible buffer known in the art in which the compound is sufficiently soluble, such as DMSO, ethanol, methanol, DMSO, DMA, DMF or water.

Typically, cultured macrophages are exposed to different concentrations of test compound (including controls exposed to the vehicle alone), for example compound may be added to replicate wells of a multi-well plate in groups of two to ten wells, more typically three to five wells. The cells may then be left exposed to the test compound for various periods from several minutes to several hours or more, typically at 37° C.

A test compound suitable for use in the present methods may be a small chemical entity, peptide, antibody molecule or other molecule whose effect on apoptotic cell phagocytosis is to be determined. Natural or synthetic chemical compounds may be used, or extracts of plants which contain several characterised or uncharacterised components.

Suitable test compounds may be selected from compound collections and designed compounds. Combinatorial library technology (Schultz, J S (1996) Biotechnol. Prog. 12:729-743) provides an efficient way of testing a potentially vast number of different substances for ability to stimulate apoptotic cell phagocytosis.

In some preferred embodiments, a suitable test compound may be a peptide fragment of a mammalian ApoE polypeptide, for example human ApoE, or an analogue, mimetic, derivative or modification thereof. For example, peptide fragments of from 5 to 40 amino acids, for example, from 6 to 10 amino acids of ApoE may be tested for ability to stimulate apoptotic cell phagocytosis.

Peptide fragments of ApoE may be produced by any method of peptide synthesis known in the art, for example by chemical synthesis or recombinant in vitro or in vivo expression.

ApoE peptides may be generated wholly or partly by chemical synthesis. An ApoE peptide as described herein can be readily prepared according to well-established, standard liquid or, preferably, solid-phase peptide synthesis methods, general descriptions of which are broadly available (see, for example, in J. M. Stewart and J. D. Young, Solid Phase Peptide Synthesis, 2nd edition, Pierce Chemical Company, Rockford, Ill. (1984), in M. Bodanzsky and A. Bodanzsky, The Practice of Peptide Synthesis, Springer Verlag, New York (1984); Applied Biosystems 430A Users Manual, ABI Inc., Foster City, Calif.; Roberge, J. Y. et al. (1995) Science 269:202-204; Caruthers, M. H. et al. (1980) Nucl. Acids Res. Symp. Ser. 215-223; Horn, T. et al. (1980) Nucl. Acids Res. Symp. Ser. 225-232; and Merrifield J. (1963) J. Am. Chem. Soc. 85:2149-2154), or may be prepared in solution, by the liquid phase method or by any combination of solid-phase, liquid phase and solution chemistry, e.g. by first completing the respective peptide portion and then, if desired and appropriate, after removal of any protecting groups being present, by introduction of the residue X by reaction of the respective carbonic or sulfonic acid or a reactive derivative thereof. Automated synthesis may be performed, for example, using the ABI 431 A Peptide Synthesizer (Applied Biosystems).

An ApoE peptide may have an amino terminal (N-terminal) capping group (e.g. an acetyl group) and/or a carboxy terminal (C-terminal) capping group, (e.g. an amide group) to protect the terminal residue from undesirable chemical reactions during use or to permit further conjugations or manipulations of the peptide. For example, the modulatory properties of a peptide fragment as described above may be increased by the addition of one of the following groups to the C terminal: chloromethyl ketone, aldehyde and boronic acid. These groups are transition state analogues for serine, cysteine and threonine proteases. The N terminus of a peptide fragment may be blocked with carbobenzyl to inhibit aminopeptidases and improve stability (Proteolytic Enzymes 2nd Ed, Edited by R. Beynon and C. Bond Oxford University Press 2001).

A newly synthesized peptide may be substantially purified by preparative high performance liquid chromatography (e.g., Creighton, T. (1983) Proteins, Structures and Molecular Principles, W H Freeman and Co., New York, N.Y.) or other comparable techniques available in the art. The composition of the synthetic peptides may be confirmed by amino acid analysis or sequencing (e.g. the Edman degradation procedure).

Recombinant in vivo or in vitro production of an ApoE peptide may be achieved by the expression of a nucleic acid that comprises an encoding nucleotide sequence using conventional recombinant techniques.

Other candidate compounds may be based on modelling the 3-dimensional structure of ApoE and using rational drug design to provide potential enhancer compounds with particular molecular shape, size and charge characteristics. Methods and means of rationale drug design are well known in the art.

The effect of a compound identified by a method described above may be assessed in a secondary screen. For example, the effect of the compound on one or more symptoms of a condition described herein may be determined in vivo in an animal model.

Control experiments may be performed as appropriate in the methods described herein. The performance of suitable controls is well within the competence and ability of a skilled person in the field.

A method as described herein may comprise identifying a test compound as an agent that stimulates, enhances or increases apoptotic cell phagocytosis.

The identified compound may be isolated and/or purified. In some embodiments, the compound may be prepared, synthesised and/or manufactured using conventional synthetic techniques.

Optionally, compounds identified as agents which stimulate apoptotic cell phagocytosis using an method described herein may be modified or subjected to rational drug design techniques to optimise activity or provide other beneficial characteristics such as increased half-life or reduced side effects upon administration to an individual.

Further optimisation or modification may then be carried out to arrive at one or more final compounds or in vivo or clinical testing.

Compound produced by the present methods described above may be formulated into a composition, such as a medicament, pharmaceutical composition or drug, with a pharmaceutically acceptable excipient, carrier, buffer, stabiliser or other materials well known to those skilled in the art.

A pharmaceutically acceptable excipient or other material should be non-toxic and should not interfere with the efficacy of the active ingredient. The precise nature of the carrier or other material will depend on the route of administration, which may be oral, or by injection, e.g. cutaneous, subcutaneous or intravenous.

A method of producing a pharmaceutical composition for use in treating a condition described herein may comprise;
identifying and/or obtaining a compound which stimulates apoptotic cell phagocytosis using a method described herein; and,
admixing the compound identified thereby with a pharmaceutically acceptable carrier.

As described above, the compound may be modified to optimise the pharmaceutical properties thereof.

A method for preparing a pharmaceutical composition for the treatment of a condition described herein may comprise;
i) identifying and/or obtaining a compound which stimulates apoptotic cell phagocytosis, for example using a method as described herein,
ii) synthesising the identified compound, and;
iii) incorporating the compound into a pharmaceutical composition.

A pharmaceutical composition may include, in addition to a compound identified as a stimulator of apoptotic cell phagocytosis, a pharmaceutically acceptable excipient, carrier, buffer, stabiliser or other materials well known to those skilled in the art.

Pharmaceutical compositions for oral administration may be in tablet, capsule, powder or liquid form. A tablet may include a solid carrier such as gelatin or an adjuvant. Liquid pharmaceutical compositions generally include a liquid carrier such as water, petroleum, animal or vegetable oils, mineral oil or synthetic oil. Physiological saline solution, dextrose or other saccharide solution or glycols such as ethylene glycol, propylene glycol or polyethylene glycol may be included.

For intravenous, cutaneous or subcutaneous injection, or injection at the site of affliction, the composition may be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, or Lactated Ringer's Injection. Preservatives, stabilisers, buffers, antioxidants and/or other additives may be included, as required.

The invention encompasses a compound identified and/or obtained using a method described above as an agent which may be useful in the treatment of a condition described herein, a pharmaceutical or veterinary composition, medicament, drug or other composition comprising such a compound, a method comprising administration of such a composition to an individual, e.g. a human or non-human animal, for treatment (which may include preventative treatment) of a condition described herein, use of such a compound in manufacture of a composition for administration, e.g. for treatment of a condition described herein, and a method of making a pharmaceutical or veterinary composition comprising admixing such a compound with an excipient, vehicle or carrier, for example a pharmaceutically acceptable excipient, vehicle or carrier, and optionally other ingredients.

Administration of the compound is preferably in a “prophylactically effective amount” or a “therapeutically effective amount” (as the case may be, although prophylaxis may be considered therapy), this being sufficient to show benefit to the individual. The actual amount administered, and rate and time-course of administration, will depend on the nature and severity of what is being treated. Prescription of treatment, e.g. decisions on dosage etc, is within the responsibility of general practitioners and other medical doctors.

Other aspects of the invention relate to the use of ApoE polypeptides and mimetics thereof to stimulate the clearance of apoptotic cells, for example in the treatment of a condition associated with apoptotic cell accumulation.

A method of treating a condition associated with apoptotic cell accumulation in an individual may comprise:
increasing the expression and/or activity of ApoE in said individual.

Conditions associated with apoptotic cell accumulation include tissue trauma, Traumatic Brain Injury, acute respiratory distress syndrome, bacterial sepsis, pancreatitis or pericarditis.

ApoE activity may be increased, for example, by administering an ApoE polypeptide or mimetic thereof to said individual.

ApoE polypeptides are described in more detail above.

An ApoE mimetic is a compound that retains the activity of ApoE in stimulating apoptotic cell phagocytosis. An ApoE mimetic may be produced, for example, by determining the particular parts of the compound that are critical and/or important in determining the target property. This may be done, for example by systematically varying the amino acid residues in the peptide, e.g. by substituting each residue in turn. These parts or residues constituting the active region of ApoE are known as its “pharmacophore”.

Once the pharmacophore has been found, its structure is modelled according to its physical properties, e.g. stereochemistry, bonding, size and/or charge, using data from a range of sources, e.g. spectroscopic techniques, X-ray diffraction data and NMR. Computational analysis, similarity mapping (which models the charge and/or volume of a pharmacophore, rather than the bonding between atoms) and other techniques can be used in this modelling process.

A template molecule is then selected onto which chemical groups that mimic the pharmacophore can be grafted. The template molecule and the chemical groups grafted on to it can conveniently be selected so that the modified compound is easy to synthesise, is likely to be pharmacologically acceptable, and does not degrade in vivo, while retaining the ability to stimulate phagocytosis of apoptotic cells. The ApoE mimetics produced by this approach can then be screened using the methods described herein to see whether they stimulate phagocytosis of apoptotic cells, or to what extent.

Other aspects of the invention provide an ApoE polypeptide or mimetic thereof for use in the treatment of a condition associated with apoptotic cell accumulation and the use of an ApoE polypeptide or mimetic thereof in the manufacture of a medicament for use in the treatment of a condition associated with apoptotic cell accumulation.

Other aspects of the invention relate to the stimulation of the clearance of apoptotic cells, for example in the treatment of a condition associated with decreased endogenous apoE of increased inflammatory activity.

A method for the treatment of a condition associated with decreased endogenous apoE or increased inflammatory activity in an individual may comprise administering a compound which stimulates phagocytosis of apoptotic cells.

The compound may be a peptide fragment of human ApoE or an analogue or derivative thereof. Suitable compounds are described in more detail above.

A condition associated with decreased endogenous apoE may be selected from the group consisting of Alzheimer's Disease, atherosclerosis, stroke and osteoporosis

A condition associated with increased inflammatory activity may be selected from the group consisting of multiple sclerosis, rheumatoid arthritis, irritable bowel syndrome, Crohn's Disease, stroke, myocardial infarction, asthma, allergic rhinitis, eczema, psoriasis and contact hypersensitivity dermatitis.

Other aspects of the invention provide a compound which stimulates phagocytosis of apoptotic cells for the treatment of a condition associated with decreased endogenous apoE or increased inflammatory activity and the use of a compound which stimulates phagocytosis of apoptotic cells in the manufacture of a medicament for the treatment of a condition associated with decreased endogenous apoE or increased inflammatory activity.

Various further aspects and embodiments of the present invention will be apparent to those skilled in the art in view of the present disclosure. All documents mentioned in this specification are incorporated herein by reference in their entirety.

The invention encompasses each and every combination and sub-combination of the features that are described above.

Certain aspects and embodiments of the invention will now be illustrated by way of example and with reference to the figures described below.

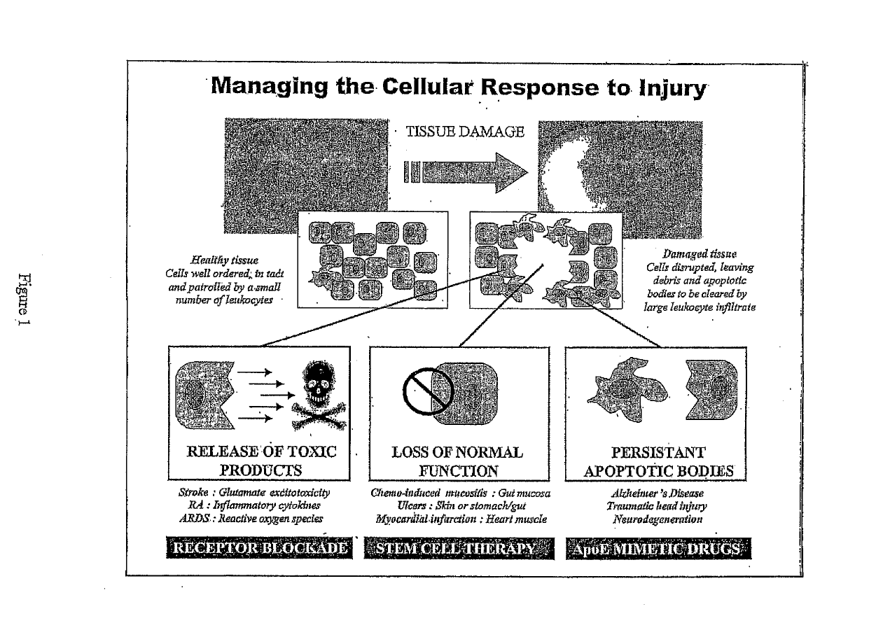

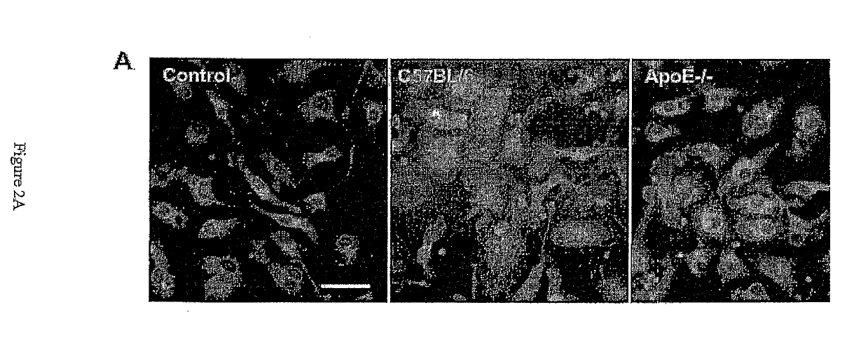

FIG. 1 shows three pathways which constitute the tissue response to injury. Following tissue injury (such as cerebral infarction during stroke, shown in the top images of a normal (left) and infarcted (right) brain stained to reveal the damaged area (white)), the body has to cope with the release of cytokines and other products from the damaged cells, the loss of function of the lost tissue and clear up the debris and apoptotic cells. This third limb of the response may be poorly active in certain individuals (such as those with certain polymorphisms in the apoE gene).

FIG. 2 shows the effect of apoE deficiency on phagocytosis in vitro. (a) Fluorescence micrographs of macrophages after taking up labeled apoptotic thymocytes for 30 minutes at 37° C. In the control image no thymocytes were added. The images were captured at high gain so that the unlabelled macrophages are visible by autofluorescence in addition to the labeled thymocytes (arrowhead). The scale bar represents 25 μm. (b) Quantitation of the number of wild-type thymocytes taken up per macrophage from images such as those presented in (a). The total number of thymocytes in ten fields of view were divided by the total number of macrophages. Values are then the mean±SEM from triplicate wells in each of three experiments. (c) Quantitation of the number of apoE-deficient thymocytes taken up per macrophage. The effect of pre-incubation with 1 μM soluble recombinant human apoE3 is also shown. (d) A typical flow cytometric histogram of macrophages after uptake of fluorescently labeled latex beads for 30 minutes at 37° C. Cells which had taken up no beads have a low fluorescence intensity (FL1-H), while cells taking up 1, 2, 3 or more beads are progressively shifted to higher fluorescence intensities. A typical histogram for wild-type macrophages is shown in black, and for apoE-deficient macrophages in red. (e) Quantitation of the number of latex beads taken up per macrophage from flow cytometric histograms such as that presented in (d) Values are the mean±SEM for triplicate wells in each of three separate experiments.

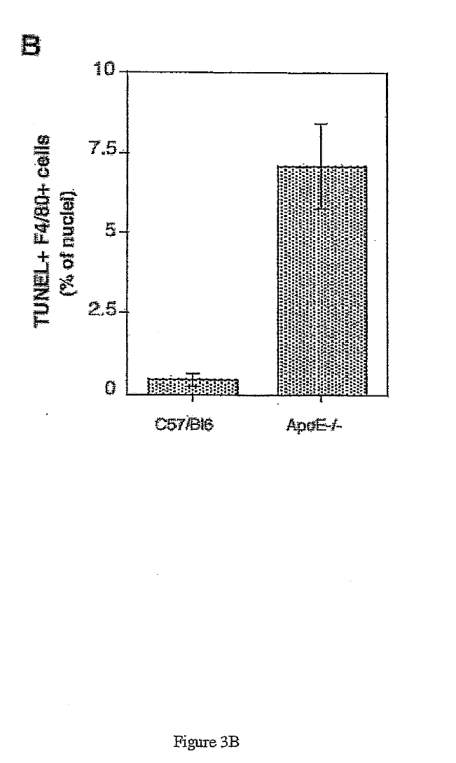

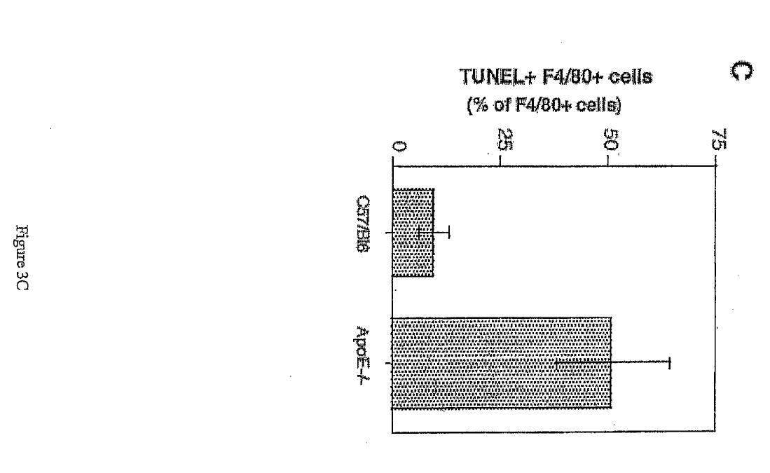

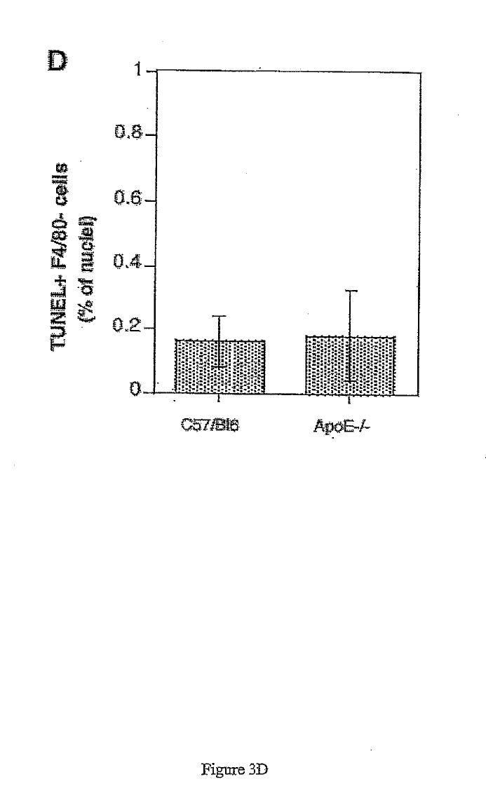

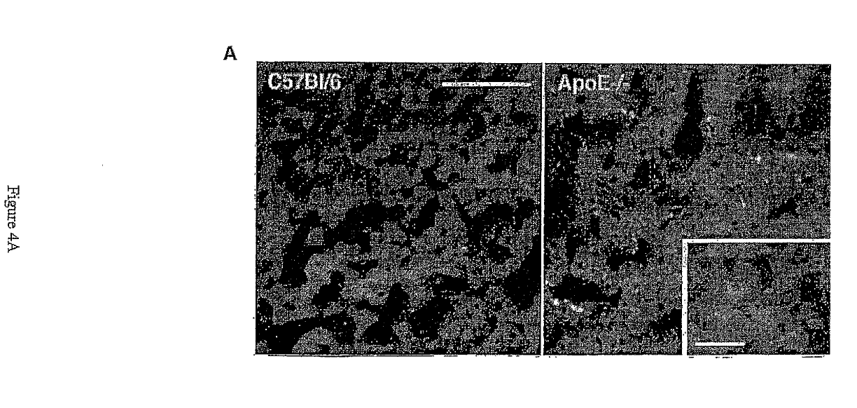

FIG. 3 shows a TUNEL staining of mouse liver. (a) Two fluorescence micrographs of the same field of view from a typical section of liver from an apoE-deficient mouse. Macrophages (mostly Küpffer cells) are illuminated in the red channel (right panel) using the monoclonal antibody F4/80. Cell remnants and dying cells are stained using the TUNEL reaction in the green channel (left panel). All of the TUNEL+ cells in this image are also F4/80+, but about 50% of the F4/80+ cells are TUNEL− (white arrows). The scale bar represents 25 μm. (b) Quantification of the absolute number of TUNEL+ F4/80+ cells in 10 sections of liver from each of 6 mice of each genotype, expressed as a percentage of the total number of nuclei analyzed. (c) The number of TUNEL+ F4/80+ cells expressed as a percentage of the number of F4/80+ cells in the same sections analyzed in (b). (d) The number of TUNEL+ F4/80− cells (that is cell remnants or dying cells which are not macrophages), expressed as a percentage of the total number of nuclei analyzed. In (b)-(d) the values are mean±SEM for 6 animals.

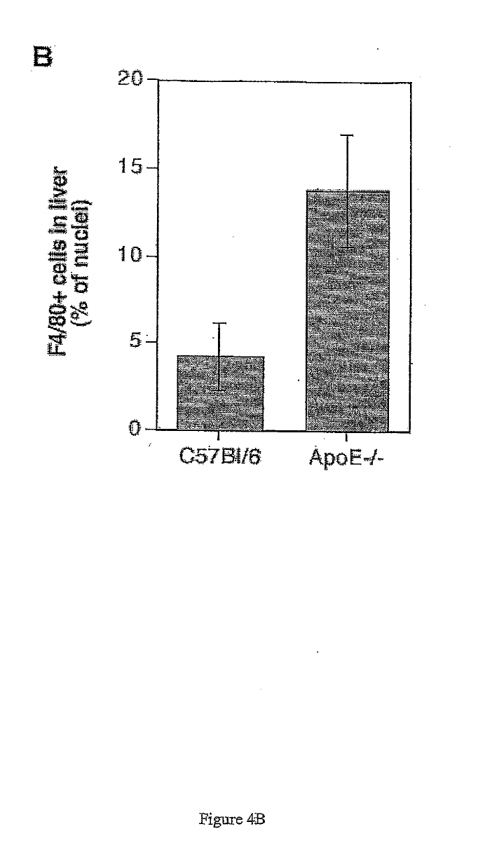

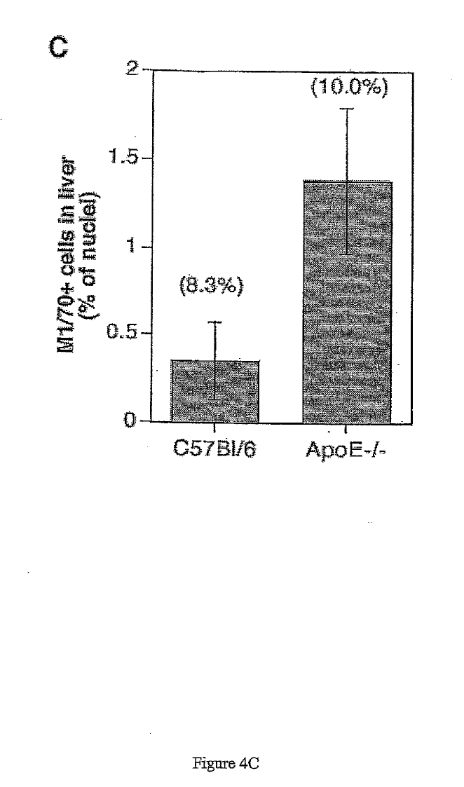

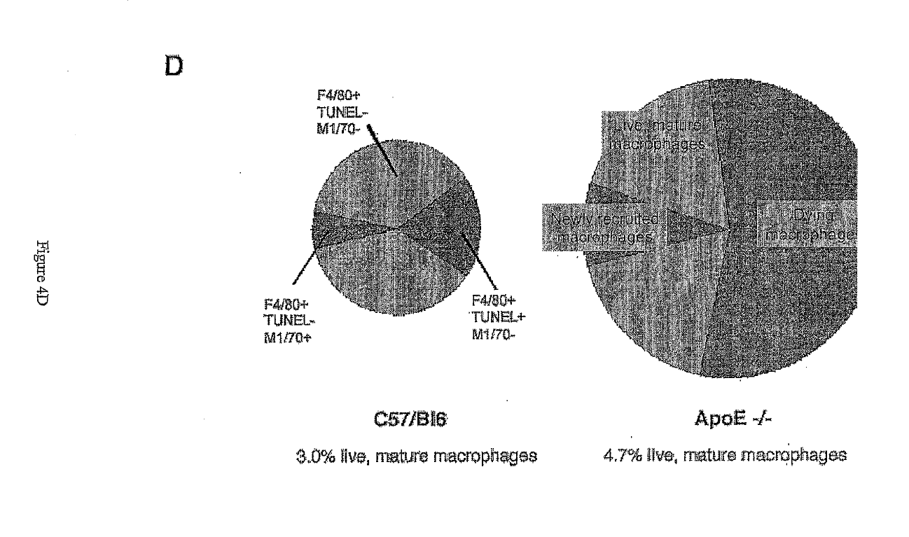

FIG. 4 shows macrophage population dynamics in mouse liver. (a) Low power fluorescence micrographs of typical liver sections from wild-type and apoE-deficient mice, stained using the F4/80 monoclonal antibody. At this magnification (scale bar represents 25 μm), the macrophages appear as white specks, which are confirmed to be specific cellular staining under high magnification (inset; scale bar represents 10 μm). (b) Quantification of the number of F4/80+ cells in 10 sections of liver from each of 6 groups of mice of each genotype, expressed as a percentage of the total number of nuclei analyzed. (c) Quantification of the number of M1/70+ cells (expressing high levels of the integrin CD11b, representing recently-recruited macrophages) in the same sections as (b). The absolute number of M1/70+ cells as percentage of the total number of nuclei analyzed is depicted, and the numbers above each bar represent the number of M1/70+ cells as a fraction of number of F4/80+ cells in the same sections. In (b) and (c), values represent the mean±SEM for 6 mice. (d) A summary of macrophage population dynamics in wild-type (left chart) and apoE-deficient (right chart) mice. The area of each chart is proportional to the total number of F4/80+ cells, and it is subdivided to show the proportions of this macrophage population which are newly recruited (M1/70+ TUNEL−; green), mature (M1/70− TUNEL−; grey) or dying (M1/70− TUNEL+; red). The number of live, mature macrophages (M1/70− F4/80+ TUNEL− cells), expressed as a percentage of the total number of nuclei, is also shown.

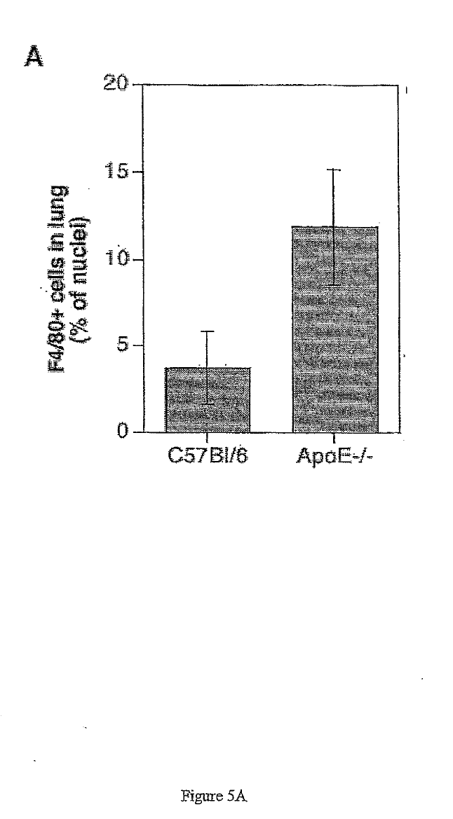

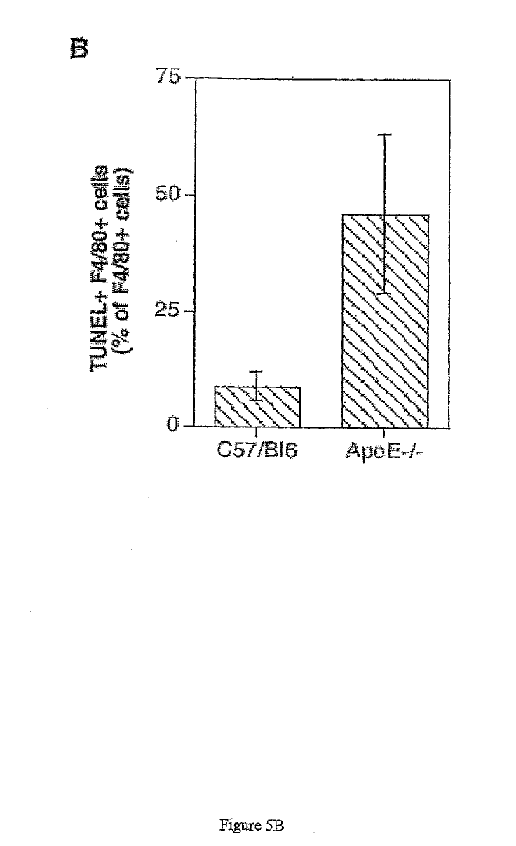

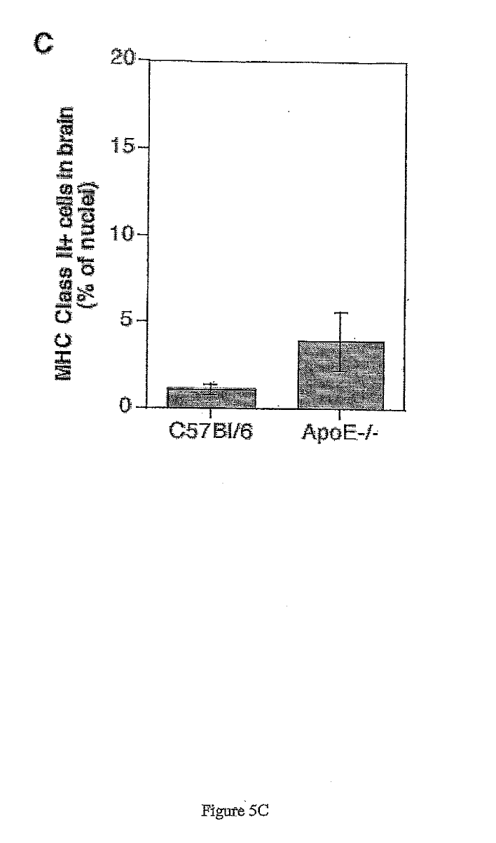

FIG. 5 shows macrophage populations in mouse lung and brain. (a) The number of F4/80+ cells (mostly alveolar macrophages) in 10 sections from the lung from 6 mice of each genotype, expressed as a percentage of the total number of nuclei analyzed. (b) The number of TUNEL+ F4/80+ cells in the same section as in (a), expressed a percentage of the number of F4/80+ cells. (c) The number of MHC Class II+ cells (predominantly microglia) in 10 sections from the brain from 6 mice of each genotype, expressed as a percentage of the total number of nuclei analyzed. (d) The number of TUNEL+ Class II+ cells in the same sections as in (c), expressed as a percentage of the number of MHC Class II+ cells. In each case, values are mean±SEM for 6 animals.

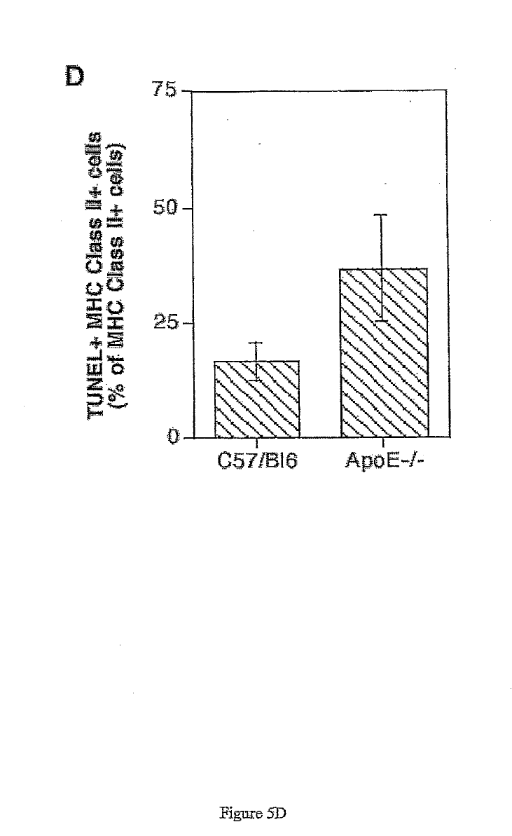

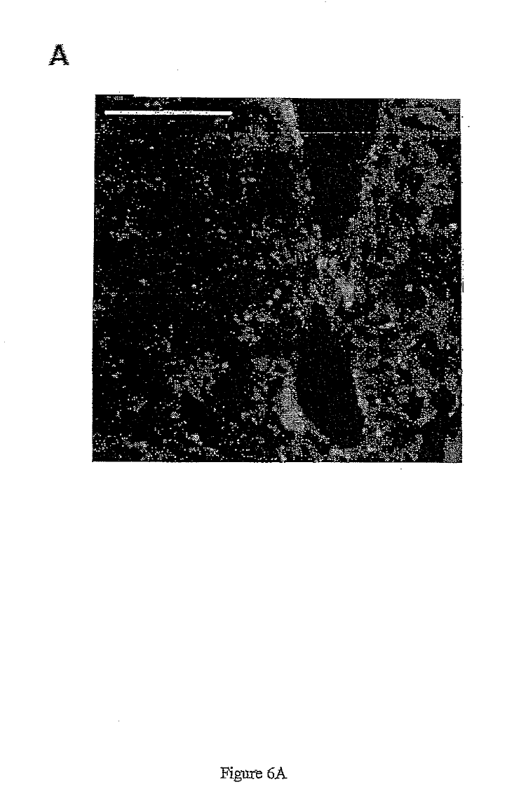

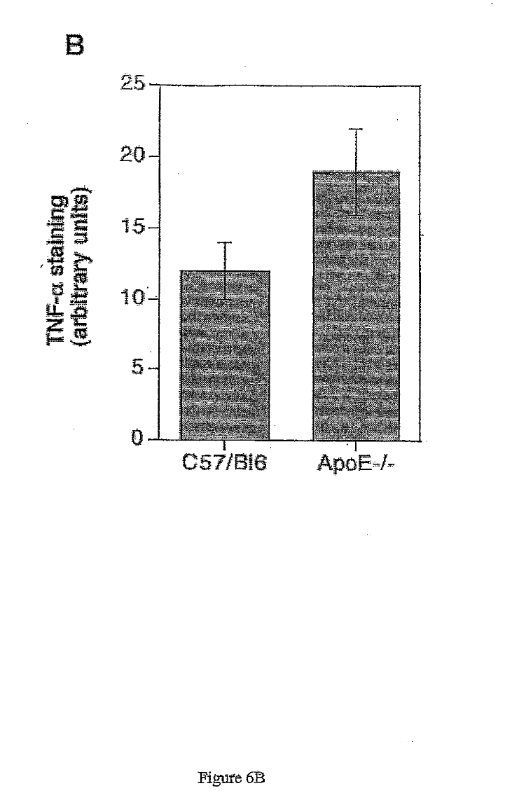

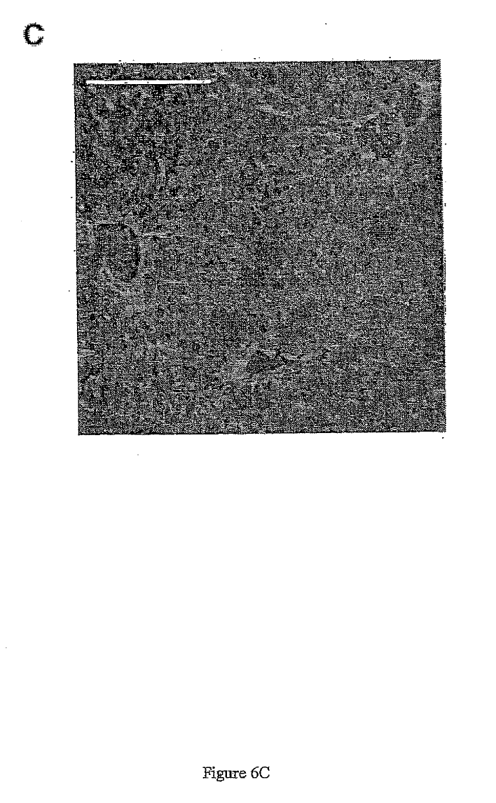

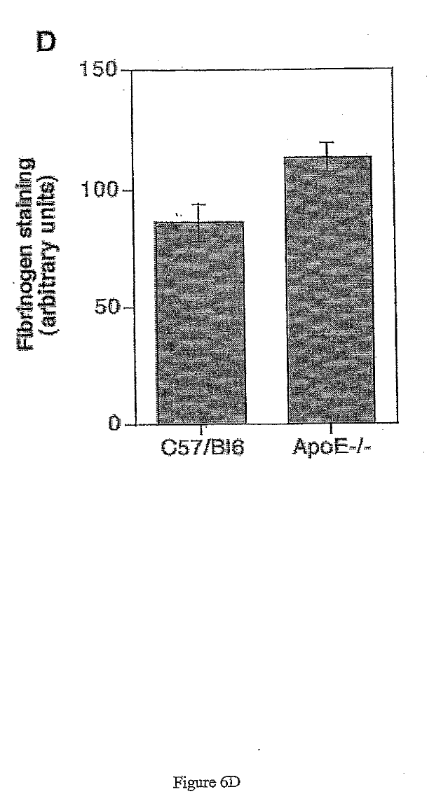

FIG. 6 shows markers of inflammation in mouse liver. (a) A fluorescence micrograph of a typical section of liver from a wild-type mouse stained for TNF-α. Most regions of the section contain little or no detectable TNF-α, but cells in the region of the small blood vessels stain strongly positive. Scale bar represents 25 μm. (b) TNF-α staining in 10 sections of liver from each of 6 mice of each genotype, measured by quantitative immunofluorescence procedures as previously described. (c) A fluorescence micrograph of a typical section of liver from a wild-type mouse stained for fibrinogen. High levels of staining are seen in the central veins of each functional unit within the liver as well as in the sinusoids. Scale bar represents 25 μm. (d) Fibrinogen staining in 10 sections of liver from each of 6 mice of each genotype, measured by quantitative immunofluorescence procedures as previously described. In each graph, values are mean±SEM for 6 mice.

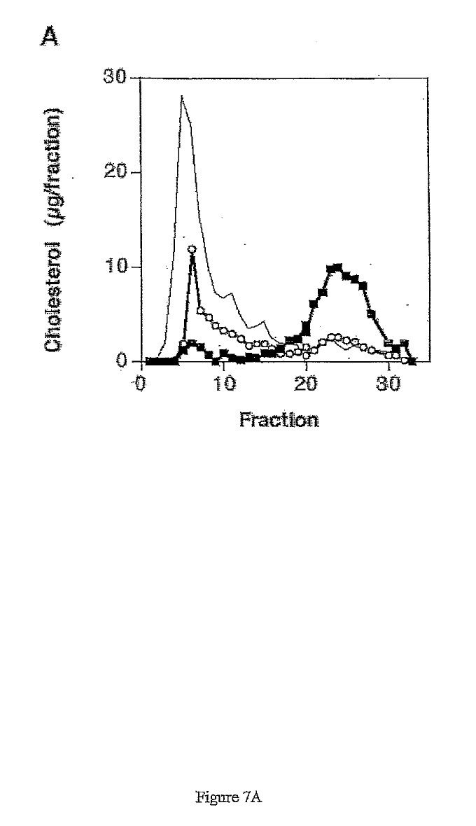

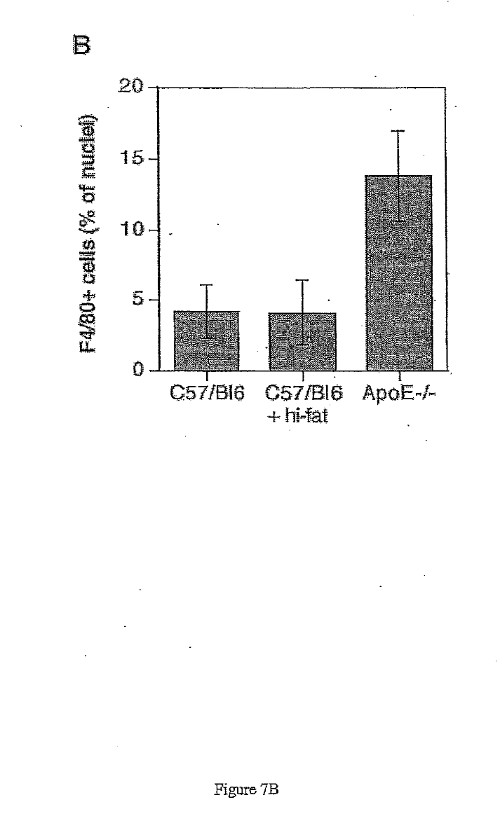

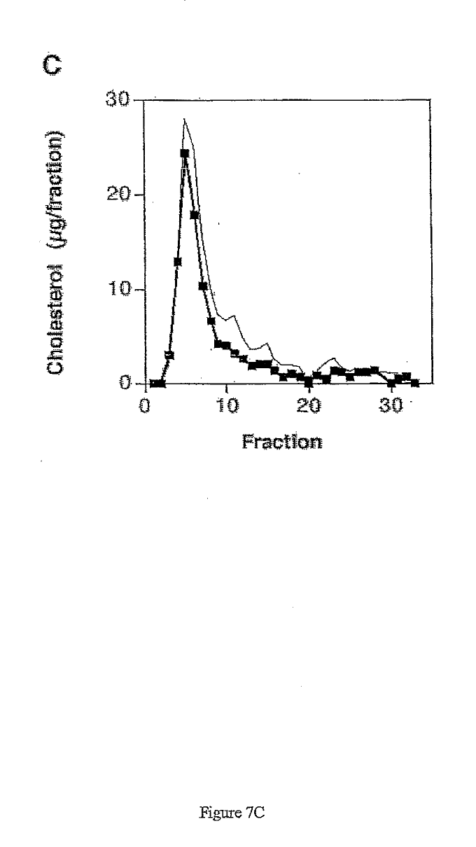

FIG. 7 shows the effect of altered lipoprotein metabolism on macrophage populations. (a) Lipoprotein profile of pooled sera from 6 wild-type mice sacrificed at 22 weeks of age, fed either a high fat diet (open circles) or normal chow diet (filled squares) for 10 weeks. The lipoprotein profile from pooled sera from 6 apoE-deficient mice is shown (plain line) for comparison. In this protocol, VLDL elutes before fraction 10, LDL between fractions 10 and 20 and HDL after fraction 20. (b) The number of F4/80+ cells, expressed as a percentage of the total number of nuclei analyzed, in 10 sections of liver from each of 6 mice in each group. (c) Lipoprotein profile of pooled sera from 6 LDL receptor-deficient mice on a normal chow diet throughout. The lipoprotein profile from pooled sera from 6 apoE-deficient mice is shown (plain line) for comparison. (d) The number of F4/80+ cells, expressed as a percentage of the total number of nuclei analyzed, in 10 sections of liver (dark bars) or lung (light bars) from each of 6 mice of each genotype. In each graph, values are mean±SEM for 6 mice.

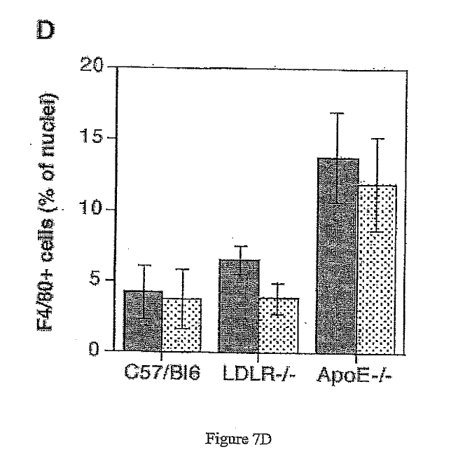

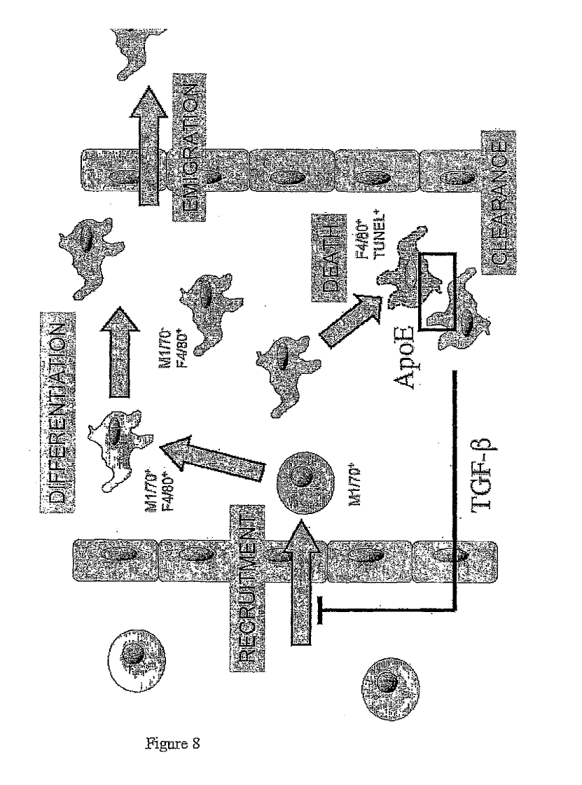

FIG. 8 shows a model of macrophage population dynamics in mouse liver. Processes increasing the equilibrium population of mature macrophages (F4/80+) include recruitment of monocytic precursors across the vascular endothelium, differentiation via M1/70+ F4/80+ newly recruited macrophage and proliferation. Processes reducing the equilibrium population of mature macrophages include emigration across the endothelium into lymph or blood, and death, either by apoptosis or necrosis (TUNEL+ F4/80+ cells; stippled pink cell) followed by clearance via phagocytosis. We demonstrated that apoE deficiency attenuates clearance of apoptotic cell remnants.

### EXPERIMENTS

### Methods and Materials

### In Vitro Phagocytosis Assays

Murine peritoneal macrophages were used for all phagocytosis assays. Macrophages were aseptically isolated from either C57Bl/6 (wild-type) or apoE−/− mice by washing out the mouse peritoneum with ice-cold sterile Hank's solution (Sigma). The peritoneal exudate cells were stored in pre-cooled sterile glass tubes and were then washed twice with RPMI medium (pelleted at 300 g for 10 min). The cell pellet was finally resuspended in RPMI+10% FCS and plated at 5×10⁵ cells per ml onto plastic tissue culture chamber slides (Nunc). The macrophages were allowed to adhere for 2 hr at 37° C. then washed with cold Dulbecco's PBS before incubation in RPMI+10% FCS until used in the phagocytosis assay between 4 and 10 days after isolation. All cells for use in phagocytosis assays were washed thoroughly and incubated under serum-free conditions to eliminate any possible effects due to the presence of bovine apoE in the FCS used for cell maintenance.

The latex bead phagocytosis assay was adapted from Ichinose [Ichinose, M. et al 1994 Cell Immunol 156:508]. Briefly, 2 μm fluorescent microspheres (carboxylate modified, Molecular Probes) in distilled water at 4.5×10⁹ beads per ml were coated with 1% BSA then sonicated for 5 minutes. Macrophage monolayers were washed with HEPES-buffered saline pH7.5 and pre-incubated for 30 minutes with 250 μl of the same solution. The BSA-coated latex beads were added to give 2.5×10⁶ beads per well then the macrophages were incubated for one hour at 37° C. during which phagocytosis occurred. Uningested beads were then removed by five vigorous washes with ice cold Dulbecco's PBS. Macrophages were then released by addition of 0.25% trypsin (Type IIS from porcine pancreas, Sigma), for two hours at 37° C. and fixed by addition of glutaraldehyde (0.5% final concentration). The number of ingested particles was measured by flow cytometry using a FACStar (Becton Dickinson) analyzing 10,000 cells per tube and the average number of particles ingested per macrophage (the phagocytic index) was calculated using FCSPress software.

The apoptotic thymocyte phagocytosis assay was adapted from the procedures described previously [Scott, R. S. et al 2001. Nature 411:207]. Thymocytes were prepared from C57Bl/6 or ApoE−/− mice by removing the thymus into RPMI+10% FCS buffered with 20 mM Tris-HCl, pH 7.2 and passing it through a cell dissociation sieve with 40 mesh screen (Sigma). The resultant cell suspension was pelleted (500 g×5 mins) and resuspended in RPMI+10% FCS at 10⁷ cells per ml. The day before the cells were used in a phagocytosis assay (3 to 9 days after putting them into culture) the thymocytes were washed three times with sterile Dulbecco's PBS and reconstituted to 5×10⁶ cells per ml then labeled with Cell Tracker Green (Molecular Probes; 2 μM final concentration) for 30 minutes at 37° C. The labelled cells were then washed in PBS, and incubated in fresh serum-free RPMI media for 30 minutes at 37° C., washed again in PBS then incubated in serum free RPMI overnight. Removal of serum yielded over 70% apoptotic thymocytes as measured by Annexin V staining and flow cytometry. Phagocytosis assays were performed as for latex beads, except that 2×10⁶ apoptotic thymocytes per well were added. Ingested thymocytes were counted by fixing the macrophages on the glass slides with 1% acetic acid in 70% ethanol for 90 minutes, and examining them under a fluorescence microscope (Provis AX; Olympus) attached to an image analysis system. The number of ingested thymocytes and the total of number of macrophages were counted in each of 18 fields of view per well and the average phagocytic index was calculated.

### Tissue Preparation

Adult male mice (either C57/Bl6, ApoE−/− or LDLR−/− mice [Ishibashi, S. et al 1994. J Clin Invest 93:1885]; six animals per group) were sacrificed by CO₂ asphyxiation and blood was drawn by cardiac puncture for preparation of serum. Tissues (left lobe of liver, left lung and left hemisphere of brain) were rapidly dissected out into ice cold saline, then embedded in OCT embedding medium and frozen at −80° C. For the animals receiving a dietary lipid challenge, normal chow was replaced with a high lipid content chow (1.25% cholesterol, 7.5% saturated fat) for ten weeks prior to sacrifice. All other animals received normal chow diet and water ad libitum throughout.

Cryosections (4 μm) were then prepared from each tissue and collected onto poly-L-lysine coated slides and fixed in ice-cold acetone for 90 seconds, air dried and frozen at −20° C. until analyzed. Transverse sections were taken from a point approximately in the middle of the tissue on the anterioposterior axis, over a distance of 4 mm. For each antigen (or group of antigens in double or triple colour immunofluorescence experiments), 16 sections spaced evenly over 4 mm (every 250 μm) were used, with 10 sections receiving primary antibody, and 6 sections (selected randomly from the 16) serving as controls with the first antibody omitted, as recommended by mosedale and colleagues [Mosedale, D. E. et al 1996. J Histochem Cytochem 44:1043].

### Immunofluorescence Staining

All immunofluorescence staining was performed as described by Mosedale et al. under the conditions optimized for quantitative immunofluorescence [Mosedale et al. vide supra]. Briefly, sections were rehydrated in PBS containing 3% fatty-acid free BSA for 30 minutes, then exposed to primary antibody in PBS/3% BSA for 18 hours. After 3×3 min washes in PBS, slides were exposed to the appropriate fluorescently-labelled secondary antibody at 25 μg/ml in PBS/3% BSA for 6 hours, except where directly labeled primaries were used.

After 3 further washes in PBS, slides were rinsed in water, air dried and mounted under Citifluor AF-1 then stored at 20° C. until analyzed using an image analysis system The following commercially available primary antibodies were used: anti-macrophage F4/80 antigen (MCAP497; Serotec; 25 g/ml; anti-CD11b (M1/7b (MCA74G); Serotec; 25 ug/ml); anti-MHC Class II (I-Ab) (MCAI500F; Serotec; 20 μg/ml); anti-fibrin(ogen) (4440-8004; Biogenesis; 20 μg/ml); anti-murine TNF-□ (AB-410-NA; R&D Systems; 50 μg/ml); anti-PCNA (M0879; Dako; 12 μg/ml). All secondary antibodies were minimum cross-reactivity donkey antibodies (Jackson Immunoresearch), All antibody solutions also contained Hoechst 33342 (1 μg/ml final concentration) to counterstain nuclei, visible on the blue channel.

### Detection of Apoptotic and Necrotic Cells

Dying cells were detected using the TUNEL reaction, as previously described [Gavrieli, Y. et al 1992. J Cell Biol 119:493], using the In Situ Cell Death Detection kit (Roche) in accordance with the manufacturer's instructions. Sections pre-treated with bovine DNase I (Roche; 10 μg/ml final concentration in 50 mM Tris pH7.5, 1 mM MgCl₂) were used as positive controls; omission of the fluorescein-labeled dUTP was used as the negative control. In mouse liver, approximately 70% of the TUNEL+ cells also stained positively for activated caspase-3 (using the CM-1 antibody) and showed signs of nuclear condensation when viewed for Hoechst 33342 fluorescence suggesting the majority of the cells detected as TUNEL+ using this kit were undergoing apoptosis, as opposed to necrosis, according to the definitions of Stadelmann & Lassmann [Stadelmann, C., and H. Lassmann. 2000. Cell Tissue Res 301:19], as claimed by the manufacturers.

### Lipoprotein Profile Analysis

Pooled serum samples were subjected to gel filtration chromatography using a Sepharose 6B column exactly as previously described [Grainger, D. J. et al 1995. Nat Med 1:1067]. Cholesterol was detected in the resulting fractions using the cholesterol oxidase method, and assigned to the various lipoprotein classes on the basis of apolipoprotein elution profiles analyzed by gel electrophoresis and Western blotting as previously described [Yokode, M. et al (1990) Science 250:1273].

### Results

Effect of apoE Deficiency on Macrophage Uptake of Apoptotic Cells In Vitro

Peritoneal macrophages were prepared from apoE-deficient mice (E−/−) and wild-type (WT) mice as controls. After 96 hours in culture, the E−/− and WT cells were each presented with fluorescently-labelled apoptotic WT thymocytes which they ingested over a period of 1 hour at 37° C. The number of thymocytes ingested by each macrophage was quantitated by immunofluorescence microscopy (FIG. 2A) as a measure of the capacity of the macrophages to clear apoptotic bodies. In this period WT macrophages each ingested 1.65±0.12 apoptotic WT thymocytes, but no ingestion was seen if live thymocytes were used (data not shown) confirming that this assay was specific for uptake of apoptotic bodies. In contrast, the E−/− macrophages ingested 25% less apoptotic thymocytes in the same period (p<0.05, Student's unpaired t-test; FIG. 2B).

Flow cytometry using an FITC-labeled antibody against murine apoE demonstrated that thymocytes express apoE, albeit at a lower level than macrophages. We therefore repeated the experiment using E−/− thymocytes. Interestingly, WT macrophages did not ingest E−/− thymocytes as efficiently as they did WT thymocytes (p<0.05, Student's t-test; FIG. 2C), and an even greater impairment in uptake of apoptotic bodies was seen when both the ingesting macrophages and the apoptotic thymocytes were apoE-deficient, with approximately 60% fewer apoptotic cells ingested than in the wild-type system (p<0.05, ANOVA; FIG. 2C). Taken together, these results demonstrate that although ingestion of apoptotic bodies can occur in the complete absence of apoE, the process is markedly attenuated.

Since ingestion of apoptotic bodies is decreased to a similar extent by deficiency of apoE on the macrophage or thymocyte partner, we tested whether exogenous addition of soluble apoE could restore normal function. Addition of human apoE3 at 1 μm (a similar concentration to human plasma) reversed the attenuation of thymocyte ingestion caused by endogenous apoE deficiency (p<0.05 versus no addition of apoE; p=0.98 versus wild-type cells, Student's unpaired t-test; FIG. 2C). We conclude that apoE is an important modulator of apoptotic body uptake but it is unlikely to function in a receptor:ligand pair participating in direct cellular interaction during uptake, but to have a more complex regulatory role possibly involving signaling at the cell surface.

Next we tested whether apoE is a broad-spectrum modulator of macrophage phagocytosis or whether it has specificity for the uptake of apoptotic cells. The uptake of fluorescently labeled latex beads, measured using flow cytometry (FIG. 2D), by E−/− macrophages was indistinguishable from that of WT macrophages (p=0.86, Student's unpaired t-test; FIG. 2E), demonstrating that the effect of apoE on phagocytosis was specific for the uptake of apoptotic bodies.

Effect of apoE Deficiency on Clearance of Apoptotic Bodies in Vivo

The number of apoptotic bodies present in vivo in apoE-deficient mice and wild-type littermate controls were analysed. Cryosections were prepared at 250 μm intervals through 4 mm of the left lobe of the liver, and dying cells and cell remnants were detected using the TUNEL reaction, which labels DNA breaks. Co-staining for the general macrophage marker F4/80 revealed that the majority (>98%) of TUNEL positive cells in both wild-type and apoE deficient liver were F4/80+ (most of the TUNEL positive cells were macrophages). It is important to note, however, that TUNEL labelling does not only stain apoptotic cells, and may stain necrotic cells or even healthy cells depending on the method of tissue preparation. However, in our sections many of the TUNEL+ cells had characteristics of apoptotic cells when viewed for Hoechst fluorescence (i.e. chromatin condensation and vacuolation of the nuclei were evident). Furthermore, 60-70% of the TUNEL+ cells also stained for active caspase-3. Taken together, these observations provide indication that TUNEL staining is a useful index of macrophage cell death in the liver, consistent with the findings of Stadelmann and Lassmann [vide supra].

The number of dying macrophages (F4/80+ TUNEL+) in the liver was dramatically increased in the apoE-deficient mice compared with the wild-type littermate controls (FIG. 3A-C). Both the absolute number of apoptotic macrophages (p<0.01, Student's unpaired t-test; FIG. 3B) and the proportion of the macrophage population which were TUNEL+ (p<0.05, Student's unpaired t-test; FIG. 3C) were increased. In wild-type animals approximately 10% of the F4/80+ cells were also TUNEL+, whereas in apoE-deficient animals more than 50% were TUNEL+. We also examined the impact of apoE-deficiency on the number of apoptotic hepatocytes (F4/80− TUNEL+ cells). However, the rate of cell death among the non-macrophage cellular compartments was very much lower than among the macrophage compartment (<2% of all the TUNEL+ cells were F/480−) so our experiment was underpowered to detect any impact of apoE-deficiency on this population (FIG. 3D).

The observed increase in apoptotic macrophages in the liver of apoE-deficient mice could have resulted from either an increased rate of macrophage death or a decreased rate of apoptotic body clearance. To distinguish these two possibilities, we analyzed the macrophage population more fully to provide a picture of macrophage population dynamics in mouse liver. First we estimated the size of the liver macrophage population by expressing the total number of F4/80+ cells as a percentage of the total number of nuclei in the liver (FIGS. 4A,B). Despite the dramatic increase in the number and proportion of TUNEL+ macrophages in apoE-deficient mice, the total liver macrophage population was significantly larger in the apoE deficient animals (p<0.05; Student's unpaired t-test; FIG. 3B). There are at least two simple explanations for this apparent paradox: either the clearance of apoptotic bodies is significantly less efficient in apoE-deficient mice, consistent with our observations in vitro, or macrophage recruitment has been increased in the absence of apoE to an even greater extent than any increase in macrophage death.

To investigate this second possibility, we exploited the observation that the integrin CD11b (detected by the monoclonal antibody M1/70), which is highly expressed on monocytes, is down-regulated following recruitment into the tissue macrophage population. As a result, the number of M1/70+ F/480+ cells in the liver can be used as a surrogate index of macrophage recruitment. ApoE-deficiency increased the absolute number of M1/70+ F4/80+ macrophages in the liver by more than 2 fold (p<0.05, Student's unpaired t-test; FIG. 4C), but this only represents a small increase in the proportion of macrophages which are newly recruited (10.0% in apoE-deficient mice, compared with 8.3% in C57/Bl6 mice). Thus, any increase in the rate of macrophage recruitment is substantially less than the large increase seen in the number of dead or dying macrophages. Since proliferation of the macrophage population is a very rare event (no PCNA+ F480+ cells were seen in the liver sections analysed), we conclude that increased macrophage recruitment is unlikely to have accounted for the increased macrophage population seen in the apoE-deficient mice.

A summary of the macrophage population dynamics in the liver from wildtype and apoE-deficient mice is shown in FIG. 4D. ApoE deficiency has significantly perturbed the homeostasis of the macrophage population, resulting in a small but statistically significant increase in macrophage recruitment, a larger increase in the number of live macrophages but the most significant effect is the dramatic increase in the number of apoptotic bodies. Based on this analysis, we conclude that clearance of apoptotic bodies is decreased in the liver of apoE-deficient mice in vivo as well as in vitro.

### Macrophage Population Dynamics in Other Tissues

Cryosections were prepared from the brain (left hemisphere) and left lung of the same mice from which liver samples had been taken. As in the liver, apoE-deficiency increased the size of the alveolar macrophage population (FIG. 5A), with the dominant factor contributing to this increase being the accumulation of dead and dying cells (FIG. 5B). The magnitude of the effects seen in the lung were very similar to those seen in liver.

It was not possible to use the same antibody markers to characterize the brain tissue macrophage population (the microglia) because they only stained weakly with the F4/80 monoclonal antibody. However, an antibody against MHC class II (which only stains a subset of the F4/80+ cells in liver and lung) stained the microglia. ApoE-deficiency increased the number of MHC class II+ cells in the brain (FIG. 5C), albeit to a lesser extent than seen for the F4/80+ population in lung and liver. However, consistent with our observations in the other tissues, both the absolute number and the proportion of MHC class II+ cells which were TUNEL+ were markedly increased (FIG. 5D). Similar results were obtained using an antibody against CD14 to detect the microglial population, although CD14 may be selectively detecting newly-recruited macrophages or activated microglia. Taken together, these observations provide indication that the attenuation of apoptotic body uptake in the absence of apoE observed in vitro has resulted in a systemic accumulation of uncleared apoptotic bodies at equilibrium in apoE-deficient mice and that there has been a small, but statistically significant, increase in macrophage recruitment to a range of tissues, likely in response to the defect in clearance of apoptotic cells.

Effect of apoE Deficiency on Markers of Inflammation in the Liver

Quantitative immunofluorescence was used to measure the relative levels of two markers of inflammation in the liver from apoE-deficient mice and their wild-type littermates. The cytokine TNF-α is upregulated during acute inflammation, but is normally present at only very low levels in healthy liver, predominantly in the perivascular regions (FIG. 6A). Staining for TNF-α was 1.5 fold higher in apoE deficient mice compared to wild type littermates (p<0.05; Mann-Whitney U-test; FIG. 6B). Although the levels of this cytokine were low in both groups, apoE deficiency led to statistically, and possibly biologically, significant change in TNF-□ levels.

Staining for the hepatocyte product fibrinogen (FIG. 6C) was also increased by apoE deficiency (p<0.05; Student's unpaired t-test; FIG. 6D). Although primarily involved in blood clotting, fibrinogen is known to be a positive acute phase reactant (i.e. a gene product whose levels are increased during acute inflammatory responses). These two unrelated markers of systemic inflammation (TNF-α and fibrinogen) are elevated in apoE-deficient mice compared to their controls.

### Effect of Plasma Cholesterol Concentration on Macrophage Population Dynamics

In vivo, apoE deficiency results in a very significant misregulation of lipoprotein metabolism. To determine whether alterations in lipoprotein metabolism might indirectly affect macrophage population dynamics, we used two strategies independent of apoE deletion to alter lipoprotein metabolism. First, we examined the impact of feeding the wild-type mice a high cholesterol diet for 10 weeks, which resulted in an increase in both plasma LDL- and VLDL-cholesterol concentrations, and a decrease in HDL-cholesterol (FIG. 7A), so that the lipoprotein profile of the fat-fed C57/Bl6 mice more closely resembled the profile of the apoE-deficient mice, as previously observed. At the end of this period, there was no difference in the liver macrophage population (p=0.94; Student's unpaired t-test versus wild-type mice on a normal chow diet; FIG. 7B). Similarly, genetic deletion of the LDL receptor had only a marginal effect on the macrophage population in liver (p=0.07; Student's unpaired t-test; FIG. 7D) and no effect on the populations in brain and lung (p=0.88, Student's t-test; FIG. 7D), despite causing a defect in lipoprotein metabolism comparable in magnitude to that seen following deletion of apoE (FIG. 7C), as observed previously. We conclude that systemic alterations in lipoprotein metabolism are unlikely to cause the changes in macrophage population dynamics seen in apoE-deficient mice, and furthermore that apoE:LDL receptor interactions are unlikely to mediate the attenuation of apoptotic body clearance we observed.

Complete deficiency of apoE protein in macrophages is shown herein to specifically attenuate the ingestion of apoptotic cells in vitro, without affecting general phagocytosis function. This defect results in a marked increase in the accumulation of apoptotic cells and fragments in a range of tissues in apoE-deficient mice in vivo, and also in a larger population of live macrophages in these tissues. This, in turn, is associated with a systemic increase in pro-inflammatory markers, including TNF-α and fibrinogen. Although genetic deletion of apoE has previously been reported to promote macrophage recruitment to the blood vessel wall [for example, Lessner, S. et al 2002. Am J Pathol 160:2145 and Reckless, J., J. C. et al 1997. Circulation 95:1542], this has been assumed to be an indirect response to local lipid deposition rather than directly a result of apoE deficiency. Alterations in lipid metabolism are shown herein not to be responsible for these effects, and the present data provides the first direct evidence for a systemic effect of apoE on tissue macrophage recruitment (FIG. 8) which is independent of lipoprotein metabolism, resulting from impaired uptake of apoptotic cell remnants.

These results represent direct evidence of a central physiological mechanism underlying the association between apoE genotype and a range of diseases with a macrophage-rich inflammatory component. ApoE is required for efficient clearance of apoptotic bodies, and the different apoE genotypes are associated with slight differences in the clearance rates for apoptotic bodies. Over time in vivo even this small decrease in apoptotic body clearance results in increased flux through the monocyte/macrophage pathway and contributes to a systemic pro-inflammatory shift in phenotype. One sequella of such a shift is a fibrotic tendency leading to the loss of functional tissue architecture that is a hallmark of Alzheimer's disease and atherosclerosis.

1-39. (canceled)

40. A method of identifying and/or obtaining a compound for the treatment of a condition associated with decreased endogenous apoE activity in an individual comprising:
determining the ingestion of apoptotic cells by a macrophage in the presence of a test compound,
wherein an increase in apoptotic cell ingestion in the presence relative to the absence of test compound is indicative that the compound may be useful in the treatment of a condition associated with decreased endogenous apoE activity.

41. A method according to claim 40 wherein the disease condition is selected from the group consisting of Alzheimer's Disease, atherosclerosis, stroke and osteoporosis.

42. A method according to claim 40 or 41 wherein ingestion is determined in the absence of ApoE.

43. A method according to claim 40 or 41 wherein ingestion is determined in the presence of ApoE.

44. A method of identifying and/or obtaining a compound for the treatment or prevention of a condition associated with increased inflammatory activity comprising:
determining the ability of an ApoE polypeptide to stimulate the ingestion of apoptotic cells by a macrophage in the presence of a test compound,
wherein an increase in apoptotic cell ingestion in the presence relative to the absence of test compound is indicative that the compound may be useful in the treatment of a condition associated with increased inflammatory activity.

45. A method of identifying and/or obtaining a compound for the treatment or prevention of a condition associated with increased inflammatory activity comprising:
determining the ingestion of one or more apoptotic ApoE deficient cells by a ApoE deficient macrophage in the presence of a test compound,
wherein an increase in apoptotic cell ingestion in the presence relative to the absence of test compound is indicative that the compound may be useful in the treatment of a condition associated with increased inflammatory activity.

46. A method according to claim 44 or 45 wherein the condition associated with increased inflammatory activity is multiple sclerosis, rheumatoid arthritis, irritable bowel syndrome, Crohn's Disease, stroke, myocardial infarction, asthma, allergic rhinitis, eczema, psoriasis or contact hypersensitivity dermatitis.

47. A method of identifying and/or obtaining a compound for the treatment of a condition associated with apoptotic cell accumulation comprising:
determining the ability of an ApoE polypeptide to stimulate the ingestion of apoptotic cells by a macrophage in the presence of a test compound,
wherein an increase in apoptotic cell ingestion in the presence relative to the absence of test compound is indicative that the compound may be useful in the treatment of a condition associated with apoptotic cell accumulation.

48. A method of identifying and/or obtaining a compound for the treatment or prevention of a condition associated with apoptotic cell accumulation comprising:
contacting an ApoE deficient macrophage with one or more apoptotic ApoE deficient cells in the presence of a test compound; and
determining the ingestion of the apoptotic cells by the macrophage,
wherein an increase in apoptotic cell ingestion in the presence relative to the absence of test compound is indicative that the compound may be useful in the treatment of a condition associated with apoptotic cell accumulation.

49. A method according to claim 47 or 48 wherein the condition is tissue trauma, Traumatic Brain Injury, acute respiratory distress syndrome, bacterial sepsis, pancreatitis or pericarditis.

50. A method according to claim 40, 44, 45, 47 or 48 wherein the test compound is a peptide fragment of human apoE or an analogue thereof.

51. A method according to claim 40, 44, 45, 47 or 48 comprising identifying a test compound as an agent which stimulates apoptotic cell phagocytosis.

52. A method according to claim 51 comprising isolating the test compound.

53. A method according to claim 51 comprising synthesising and/or manufacturing the test compound.

54. A method according to claim 51 comprising modifying the compound to optimise the pharmaceutical properties thereof.

55. A method according to claim 51 comprising formulating the compound into a composition with a pharmaceutically acceptable excipient

56. A method of producing a pharmaceutical composition for use in treating a condition associated with decreased endogenous apoE activity, increased inflammatory activity or accumulation of apoptotic cells comprising;
identifying and/or obtaining a compound which stimulates apoptotic cell phagocytosis using a method according to claim 40, and;
admixing the compound identified thereby with a pharmaceutically acceptable carrier.

57. A method of treating a condition associated with apoptotic cell accumulation in an individual which comprises:
increasing the expression and/or activity of ApoE in said individual.

58. A method according to claim 57 wherein ApoE activity is increased by administering an ApoE polypeptide or mimetic thereof to said individual.

59. A method according to claim 58 wherein the ApoE polypeptide is a peptide fragment of human ApoE.

60. A method according to claim 57 wherein the condition associated with apoptotic cell accumulation is selected from the group consisting of tissue trauma, traumatic brain injury, acute respiratory distress syndrome, bacterial sepsis, pancreatitis or pericarditis.

61. A method to treat a condition associated with apoptotic cell accumulation comprising administering an ApoE polypeptide or mimetic thereof.

62. The method according to claim 61 wherein the ApoE polypeptide is a peptide fragment of human ApoE.

63. A method for the treatment of a condition associated with decreased endogenous apoE in an individual comprising administering a compound which stimulates phagocytosis of apoptotic cells.

64. A method according to claim 63 wherein the compound is a peptide fragment of human ApoE or an analogue or derivative thereof.

65. A method according to claim 63 or claim 64 wherein the disease condition is selected from the group consisting of Alzheimer's Disease, atherosclerosis, stroke and osteoporosis.

66. A method to treat a condition associated with decreased endogenous apoE comprising administering a compound that stimulates phagocytosis of apoptotic cells.

67. The method according to claim 66 wherein the compound is a peptide fragment of human ApoE or an analogue or derivative thereof.

68. The method according to claim 66 or claim 67 wherein the disease condition is selected from the group consisting of Alzheimer's Disease, atherosclerosis, stroke and osteoporosis

69. A method for the treatment of a condition associated with increased inflammatory activity in an individual comprising administering a compound which stimulates phagocytosis of apoptotic cells.

70. A method according to claim 69 wherein the compound is a peptide fragment of human ApoE or an analogue or derivative thereof.

71. A method according to claim 69 or claim 70 wherein the condition associated with increased inflammatory activity is multiple sclerosis, rheumatoid arthritis, irritable bowel syndrome, Crohn's Disease, stroke, myocardial infarction, asthma, allergic rhinitis, eczema, psoriasis or contact hypersensitivity dermatitis.

72. A method to treat a condition associated with increased inflammatory activity comprising administering a compound that stimulates phagocytosis of apoptotic cells.

73. The method according to claim 72 wherein the compound is a peptide fragment of human ApoE.

74. The method according to claim 72 or claim 73 wherein the condition associated with increased inflammatory activity is multiple sclerosis, rheumatoid arthritis, irritable bowel syndrome, Crohn's Disease, stroke, myocardial infarction, asthma, allergic rhinitis, eczema, psoriasis or contact hypersensitivity dermatitis.

75. A method to identify a compound which modulates the efficiency of phagocytosis, comprising:
(a) exposing cultured macrophages to an apoE-derived peptide, mimetic or other test compound;
(b) contacting said macrophages with a suspension of apoptotic cells; and
(c) determining the number of apoptotic cells ingested by the macrophages.
